(19) United States
(12) Patent Application Publication
(10) Pub. No.: US 2022/0008641 A1
(43) Pub. Date: Jan. 13, 2022

(54) NON-COLLAPSING NEGATIVE PRESSURE WOUND DRESSING

(71) Applicant: KCI LICENSING, INC., San Antonio, TX (US)

(72) Inventors: Benjamin A. PRATT, Wimborne (GB); Christopher Brian LOCKE, Bournemouth (GB)

(21) Appl. No.: 17/294,624

(22) PCT Filed: Nov. 18, 2019
(86) PCT No.: PCT/US2019/062000

§ 371 Date: May 17, 2021

Related U.S. Application Data
(60) Provisional application No. 62/770,144, filed on Nov. 20, 2018.

(51) Int. Cl. A61M 1/00; A61F 13/00; A61F 13/02

(52) U.S. Cl. CPC A61M 1/915; A61F 13/00068; A61F 2013/15073; A61F 13/0209; A61F 13/00063

(57) ABSTRACT

A wound treatment system for treating a surgical wound includes a dressing. The dressing includes a manifold layer and an absorbent pouch assembly coupled to the manifold layer. The absorbent pouch assembly includes an absorbent material contained within a pouch. The dressing also includes a drape coupled to the absorbent pouch assembly and configured to be sealable over the surgical wound. The absorbent pouch assembly is positioned between the drape and the manifold layer. The wound treatment system also includes a pump fluidly communicable with the dressing and configured to draw a negative pressure at the manifold layer. The manifold layer is configured to substantially prevent medial collapse of the manifold layer under the negative pressure.

### CROSS-REFERENCE TO RELATED APPLICATIONS

This application claims the benefit of priority to U.S. Provisional Application No. 62/770,144, filed on Nov. 20, 2018, which is incorporated herein by reference in its entirety.

### BACKGROUND

The present disclosure relates to dressings for treating surgical wounds, for example incisions made during surgical operations. Such surgical wounds are typically closed (sutured, stitched, stapled, glued, etc.) along a seam. Dressings are typically applied to protect the wound and provide various therapies to the wound in order to facilitate wound healing.

One type of wound therapy that may be used with a dressing is negative pressure therapy. In negative pressure therapy, a pump is used with a dressing to pump air out of the dressing (i.e., out of a volume sealed between an external surface of the dressing and the wound) to create a negative pressure at the wound (i.e., to reduce the pressure below atmospheric pressure). Typically, such dressings collapse medially (i.e., towards a center point or centerline of the dressing) under pressure, as the pressure causes the dressing materials to compress inwardly. This medial collapse may create forces on the surgical wound, including closure forces on a surgical wound that may be desirable for some indications. However, in some cases a caregiver or patient may prefer that negative pressure be applied to a wound without application of such forces. Accordingly, a need exists for a dressing suitable for negative pressure wound therapy for surgical wounds without medial collapse of the dressing.

### BRIEF DESCRIPTION

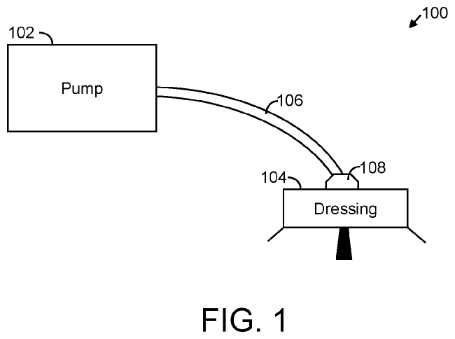

FIG. 1 is a schematic illustration of a negative pressure therapy system, according to an exemplary embodiment.

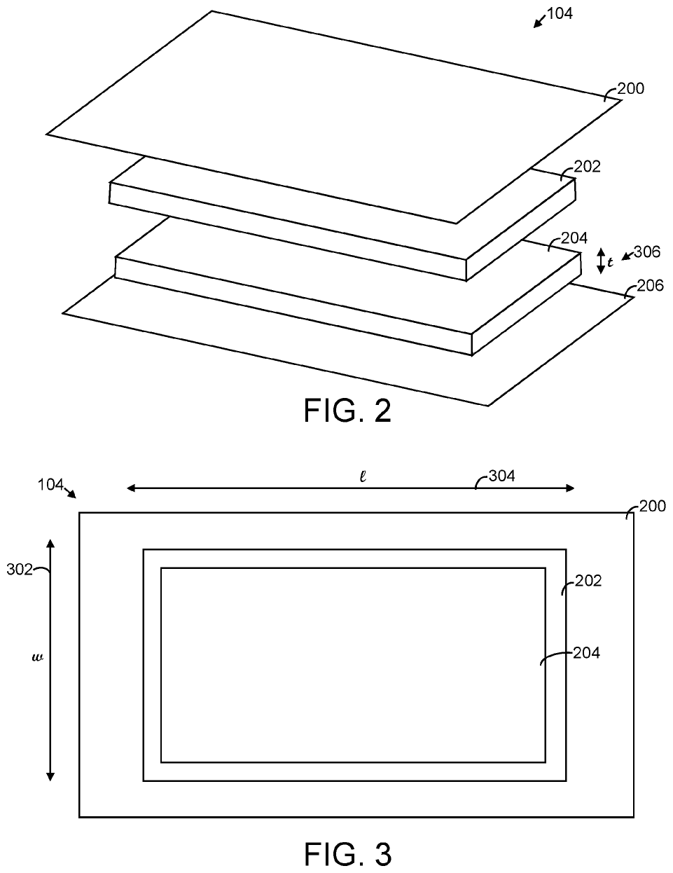

FIG. 2 is a perspective exploded view of a first embodiment of a dressing for use with the negative pressure therapy system of FIG. 1, according to an exemplary embodiment.

FIG. 3 is a bottom view of the dressing of FIG. 2, according to an exemplary embodiment.

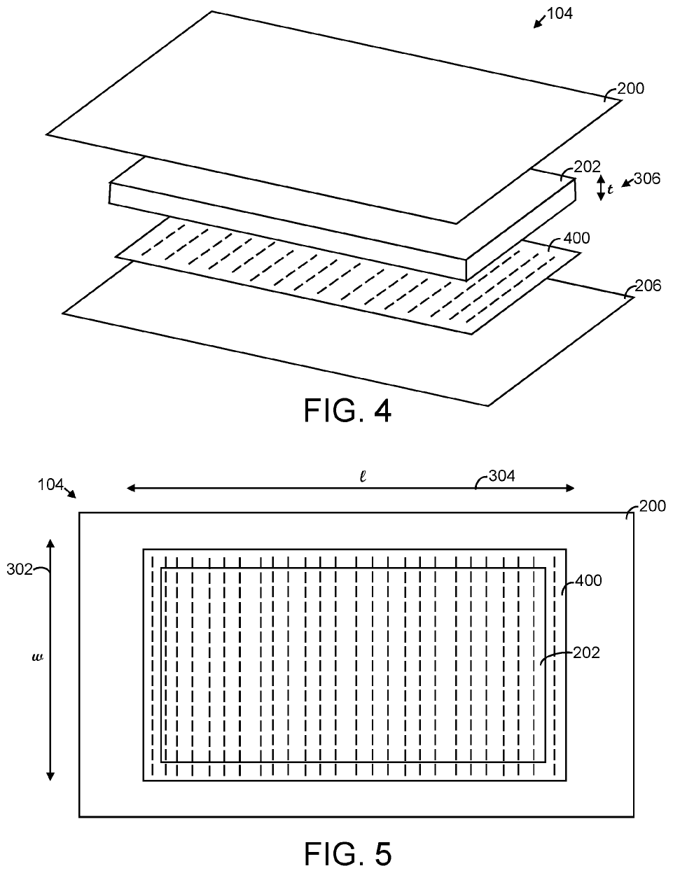

FIG. 4 is a perspective exploded view of a second embodiment of a dressing for use with the negative pressure therapy system of FIG. 1, according to an exemplary embodiment.

FIG. 5 is a bottom view of the dressing of FIG. 4, according to an exemplary embodiment.

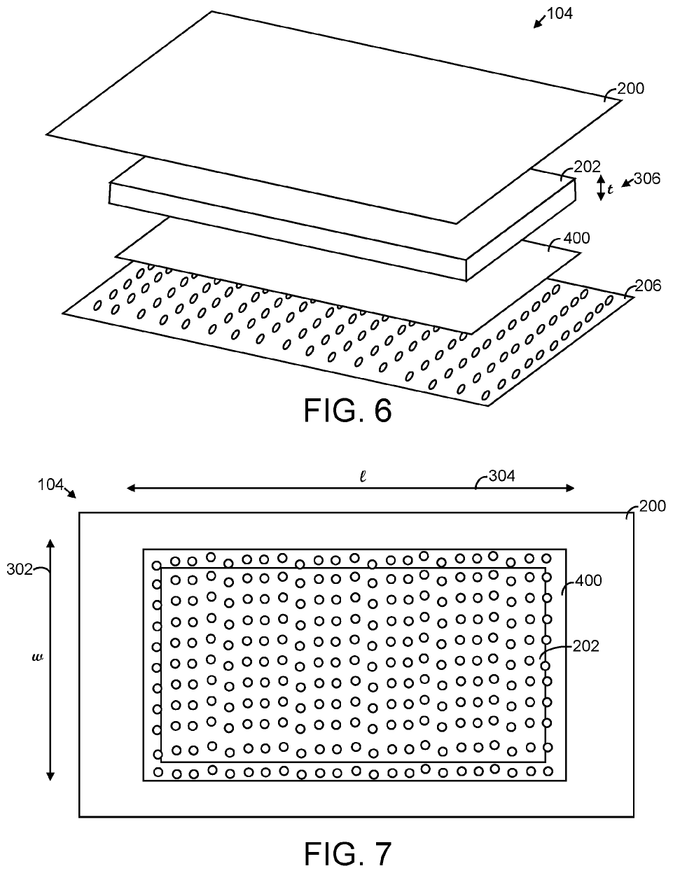

FIG. 6 is perspective exploded view of a third embodiment of a dressing for use with the negative pressure therapy system of FIG. 1, according to an exemplary embodiment.

FIG. 7 is a bottom view of the dressing of FIG. 6, according to an exemplary embodiment.

### SUMMARY

One implementation of the present disclosure is a wound treatment system for treating a surgical wound. The wound treatment system includes a dressing. The dressing includes a manifold layer and an absorbent pouch assembly coupled to the manifold layer. The absorbent pouch assembly includes an absorbent material contained within a pouch. The dressing also includes a drape coupled to the absorbent pouch assembly and configured to be sealable over the surgical wound. The absorbent pouch assembly is positioned between the drape and the manifold layer. The wound treatment system also includes a pump fluidly communicable with the dressing and configured to draw a negative pressure at the manifold layer. The manifold layer is configured to substantially prevent medial collapse of the manifold layer under the negative pressure.

In some embodiments, the manifold layer has a thickness-to-width-to-length ratio of approximately 1:10:25. In some embodiments, the thickness-to-width-to-length ratio substantially prevents the medial collapse of the manifold layer under the negative pressure.

In some embodiments, the manifold layer has a thickness-to-area ratio of approximately 1:1600. In some embodiments, the thickness-to-area ratio substantially prevents the medial collapse of the manifolding layer under the negative pressure.

In some embodiments, the dressing includes a release liner removable from the dressing. The release liner protects the manifold layer before the dressing is applied to the surgical wound.

In some embodiments, the wound treatment system includes a reduced-pressure interface integrated with the drape and aligned with a hole extending through the drape and a tube coupled between the reduced-pressure interface and the pump. The pump is fluidly communicable with the absorbent pouch assembly and the manifold layer via the tube, the reduced-pressure interface, and the hole.

In some embodiments, the manifold layer includes silver ions. In some embodiments, the manifold layer is configured to wick fluid from the surgical wound and the absorbent pouch assembly is configured to absorb the fluid from the manifold layer. In some embodiments, the absorbent material includes a superabsorbent material.

Another implementation of the present disclosure is a wound treatment system for treating a surgical wound. The wound treatment system includes a dressing. The dressing includes a wound contact film layer and an absorbent pouch assembly coupled to the wound contract film layer. The absorbent pouch assembly includes an absorbent material contained within a pouch. The dressing also includes a drape coupled to the absorbent pouch assembly and sealable over the surgical wound. The absorbent pouch assembly is positioned between the drape and the wound contact film layer. The wound treatment system also includes a pump fluidly communicable with the dressing and configured to draw a negative pressure at the surgical wound. The absorbent pouch assembly is configured to facilitate the distribution of negative pressure across the surgical wound without medial collapse of the absorbent pouch assembly.

In some embodiments, the absorbent pouch assembly comprises a manifold layer having a thickness-to-area ratio below a predetermined limit to substantial prevent the medial collapse. In some embodiments, the predetermined limit is approximately 1:800.

In some embodiments, the wound contact film layer includes a plurality of fenestrations extending therethrough. In some embodiments, the wound contact film layer includes a plurality of holes extending therethrough.

In some embodiments, the wound contact film layer allows air to flow through the wound contact film layer and the pouch allows air to flow through the pouch such that the surgical wound and the absorbent pouch assembly are maintained at substantially the same pressure.

Another implementation of the present disclosure is a method for manufacturing a dressing deployable to treat a surgical wound using negative pressure therapy without exerting a closure force on the surgical wound. The method includes enclosing an absorbent material in a pouch and coupling a manifold layer to the pouch. The manifold layer is configured to substantially prevent medial collapse of the manifold layer when subjected to a negative pressure. The method also includes coupling a drape to the pouch such that the pouch and the absorbent material are between the manifold layer and the drape and coupling a reduced-pressure interface to the drape. The reduced-pressure interface is coupleable to a pump configured to provide the negative pressure at the manifold layer.

In some embodiments, the method includes forming the manifold layer with a thickness-to-width-to-length ratio of approximately 1:10:25. In some embodiments, the method includes forming the manifold layer with a thickness-to-area ratio of approximately 1:1600. In some embodiments, the method includes forming the manifold layer with a thickness of approximately 6 millimeters.

In some embodiments, the method includes providing the manifold layer with silver ions. In some embodiments, the absorbent material is a superabsorbent material.

### DETAILED DESCRIPTION

Referring now to FIG. 1, a negative pressure therapy (NPT) system 100 is shown, according to an exemplary embodiment. The NPT system 100 includes a pump 102 fluidly communicable with a dressing 104 via a tube 106. A reduced-pressure interface (connection pad) 108 is coupled to the dressing 104 (e.g., integrated with the dressing 104). The reduced-pressure interface is configured to couple the tube 106 to the dressing 104 to place the tube 106 and pump 102 in fluid communication with the dressing 104 (i.e., such that air and/or fluid may flow from the dressing 104 to the pump 102).

As shown in FIG. 1, the dressing 104 is applied to a wound, in particular a surgical wound closed along a suture line. Various embodiments of the dressing 104 are shown in FIGS. 2-7 and described in detail with reference thereto below. As described below, the dressing 104 is sealable over the surgical wound to substantially prevent air from leaking between the dressing 104 and the patient (i.e., a periwound area around the wound). The pump 102 is operable to remove air from the dressing 104 via the reduced-pressure interface 108, thereby creating a negative pressure (relative to atmospheric pressure) at the wound. Negative pressure may provide various therapeutic advantages to the wound.

When negative pressure is applied at the dressing 104 by the pump, the pressure differential between the enclosed, negative-pressure area and the atmospheric pressure in the environment may create forces on the dressing 104 that tend to compress the dressing 104. As described in detail below, the dressing 104 is configured to facilitate distribution of negative pressure over the wound substantially without medial collapse of the dressing.

Referring now to FIGS. 2-3, detailed views of the dressing 104 are shown, according to a first embodiment. FIG. 2 shows a perspective exploded view of the dressing 104. FIG. 3 shows a bottom view of the dressing 104 (i.e., a view of the wound-facing side of the dressing 104).

As shown in FIGS. 2-3, the dressing 104 includes a drape 200, an absorbent pouch assembly 202 coupled to the drape 200, and a manifolding foam layer (manifold layer) 204 coupled to the absorbent pouch assembly 202. The absorbent pouch assembly 202 is positioned between the drape 200 and the manifolding foam layer 204. The absorbent pouch assembly 202 is aligned with the manifolding foam layer 204. As illustrated in FIG. 1, a reduced-pressure interface 108 may be coupled to the drape 200 and/or integrated with the drape 200 to place the absorbent pouch assembly 202 and/or the manifolding foam layer 204 in fluid communication with a pump 102 via a tube 106.

The dressing 104 is also shown to include a release liner 206 removably coupled to the drape 200 such that the manifolding foam layer 204 and the absorbent pouch assembly 202 are enclosed between the release liner 206 and the drape 200. The release liner 206 protects the manifolding foam layer 204 and the absorbent pouch assembly 202 from external contaminants before application of the dressing 104 to a wound (during storage, transit, etc.). FIG. 3 shows the dressing 104 without the release liner 206.

The drape 200 is sealable over the wound and configured to provide a substantially air-tight volume between the drape 200 and the wound. The drape 200 may be made of polyurethane. The drape 200 may include an adhesive border configured to removably adhere the drape 200 to a patient's skin around a wound. The drape 200 may have a larger area than the manifolding foam layer 204 and the absorbent pouch assembly 202, for example such that the adhesive border of the drape 200 surrounds the manifolding foam layer 204 and the absorbent pouch assembly 202.

The absorbent pouch assembly 202 is configured to absorb fluid exuded by the wound and retain the fluid. In some embodiments, the absorbent pouch assembly 202 allows air to flow therethrough, facilitating distribution of pressure across the dressing 104. The absorbent pouch assembly 202 may include an absorbent material contained within a pouch. The absorbent material may include an absorbent commercially-available from Gelok International Corporation. In some embodiments, the absorbent material is a superabsorbent material. The pouch may be made of a non-woven material, for example as commercially available from Libeltex BVBA. In some alternative embodiments, the absorbent pouch assembly 202 is a superabsorbent laminate, for example as described in U.S. Patent Application No. 62/788,036 filed on Jan. 3, 2019, incorporated by reference herein in its entirety. In some alternative embodiments, the absorbent pouch assembly 202 is replaced by a superabsorbent slurry deposited in a pattern on the manifolding foam layer 204.

The manifolding foam layer 204 is configured to contact the wound and facilitate the distribution of negative pressure across the wound. The manifolding foam layer 204 may include an open-cell foam that allows air to flow therethrough. The manifolding foam layer 204 may also be configured to wick fluid from the wound to the absorbent pouch assembly 202 to facilitate the absorbent pouch assembly 202 in absorbing fluid from the wound. The manifolding foam layer 204 may include silver ions or another anti-microbial substance.

The manifolding foam layer 204 is configured to substantially prevent medial collapse of the manifolding foam layer 204 and the dressing 104 under negative pressure. For example, as illustrated by FIG. 3, the manifolding foam layer 204 may be formed with a width-to-length-to-thickness ratio that substantially prevents medial collapse of the manifolding foam layer. In such a case, the width dimension 302 and length dimension 304 are substantially parallel to the wound bed, while the thickness dimension 306 is substantially perpendicular to the wound bed. In some embodiments, the width-to-length-to-thickness ratio is approximately 1:10:25. In some embodiments, the width-to-length-to-thickness ratio may be in the range of 1:(5-15):(15-25). For example, in one embodiment the width of the manifolding foam layer 204 is approximately 63.5 mm, the length is approximately 152.4 mm, and the thickness is approximately 6 mm.

As another example, the manifolding foam layer 204 may be formed with a thickness-to-area ratio. The thickness dimension 306 is substantially perpendicular to the wound bed while the area is measured as the surface area of a surface of the manifolding foam layer 204 parallel to the wound bed (i.e., as shown from the bottom view of FIG. 3). In some embodiments, the thickness-to-area ratio is approximately 1:1600. In various embodiments, the thickness-to-area ratio may be in a range of 1:(1000-2000). For example, in one embodiment the thickness of the manifolding foam layer 204 is approximately 6 mm and the area of the manifolding foam layer is approximately 9,677 square mm.

The manifolding foam layer 204 thereby substantially prevents medial collapse of the dressing 104, thereby substantially preventing medial forces from being applied to the wound while also facilitating the distribution of negative pressure across the wound. The dressing 104 is thereby configured to satisfy a caregiver's desire to treat a wound with negative pressure without the additional effects of various other forces that are created by existing dressings.

Referring now to FIGS. 4-5, a second embodiment of the dressing 104 is shown, according to an exemplary embodiment. FIG. 4 shows an exploded perspective view of the dressing 104, while FIG. 5 shows a bottom view of the dressing 104. In the embodiment shown in FIGS. 4-5, the dressing 104 includes the drape 200, the absorbent pouch assembly 202, the release liner 208, and a wound contact film layer 400.

The wound contact film layer 400 is configured to contact a wound and substantially prevent adherence of the dressing 104 to the wound. The wound contact film layer 400 is configured to allow the flow of air and fluid therethrough. In FIGS. 4-5, the wound contact film layer 400 is shown a fenestrated film with a large number of fenestrations (slits) extending therethrough. The wound and the absorbent pouch assembly may thereby be maintained at substantially the same pressure. The wound contact film layer 400 may include polyurethane and/or silicone.

In the embodiment shown in FIGS. 4-5, the wound contact film layer 400 is substantially uncollapsible under negative pressure. Furthermore, in various embodiments the absorbent pouch assembly 204 may be configured to substantially prevent medial collapse of the absorbent pouch assembly 204 and the dressing 104. In some such embodiments, a thickness-to-area ratio of the absorbent pouch assembly 204 substantially prevents medial collapse of the absorbent pouch assembly 204, for example when the thickness-to-area ration of the absorbent pouch assembly is below a predetermined limit. In some embodiments, the predetermined limit is 1:800.

Referring now to FIGS. 6-7, a third embodiment of the dressing 104 is shown, according to an exemplary embodiment. FIG. 6 shows an exploded perspective view of the dressing 104 and FIG. 7 shows a bottom view of the dressing 104. As in FIGS. 4-5, the dressing 104 as shown in FIGS. 6-7 includes the drape 200, the absorbent pouch assembly 202, the wound contact film layer 400, and the release liner 208. The dressing 104 of FIGS. 6-7 may be substantially similar to the dressing 104 of FIGS. 4-5. FIGS. 4-5 illustrated that the wound contact film layer 400 may include a perforated film with a large number of small circular holes extending therethrough. The holes may allow air and/or fluid to flow through the wound contact film layer 400.

The dressing 104 is thereby configured to facilitate the distribution of negative pressure across the dressing 104 while also substantially preventing medial collapse of the dressing 104.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. All such variations are within the scope of the disclosure.

What is claimed is:

1. A wound treatment system for treating a surgical wound, comprising:
a dressing, comprising:
a manifold layer;
an absorbent pouch assembly coupled to the manifold layer, the absorbent pouch assembly comprising an absorbent material contained within a pouch;
a drape coupled to the absorbent pouch assembly and configured to be sealable over the surgical wound, the absorbent pouch assembly positioned between the drape and the manifold layer;
a pump fluidly communicable with the dressing and configured to draw a negative pressure at the manifold layer;
wherein the manifold layer is configured to substantially prevent medial collapse of the manifold layer under the negative pressure.

2. The wound treatment system of claim 1, wherein the manifold layer has a thickness-to-width-to-length ratio of approximately 1:10:25.

3. The wound treatment system of claim 2, wherein the thickness-to-width-to-length ratio substantially prevents the medial collapse of the manifold layer under the negative pressure.

4. The wound treatment system of claim 1, wherein the manifold layer has a thickness-to-area ratio of approximately 1:1600.

5. The wound treatment system of claim 4, wherein the thickness-to-area ratio substantially prevents the medial collapse of the manifolding layer under the negative pressure.

6. The wound treatment system of claim 1, the dressing comprising a release liner removable from the dressing, the release liner protecting the manifold layer before the dressing is applied to the surgical wound.

7. The wound treatment system of claim 1, comprising:
a reduced-pressure interface integrated with the drape and aligned with a hole extending through the drape;
a tube coupled between the reduced-pressure interface and the pump;
wherein the pump is fluidly communicable with the absorbent pouch assembly and the manifold layer via the tube, the reduced-pressure interface, and the hole.

8. The wound treatment system of claim 1, wherein the manifold layer comprises silver ions.

9. The wound treatment system of claim 1, wherein the manifold layer is configured to wick fluid from the surgical wound; and
wherein the absorbent pouch assembly is configured to absorb the fluid from the manifold layer.

10. The wound treatment system of claim 1, wherein the absorbent material comprises a superabsorbent material.

11. A wound treatment system for treating a surgical wound, comprising:
a dressing, comprising:
a wound contact film layer;
an absorbent pouch assembly coupled to the wound contact film layer, the absorbent pouch assembly comprising an absorbent material contained within a pouch;
a drape coupled to the absorbent pouch assembly and sealable over the surgical wound, the absorbent pouch assembly positioned between the drape and the wound contact film layer;
a pump fluidly communicable with the dressing and configured to draw a negative pressure at the surgical wound;
wherein the absorbent pouch assembly is configured to facilitate the distribution of negative pressure across the surgical wound without medial collapse of the absorbent pouch assembly.

12. The wound treatment system of claim 11, wherein the absorbent pouch assembly comprises a manifold layer having a thickness-to-area ratio below a predetermined limit to substantial prevent the medial collapse.

13. The wound treatment system of claim 12, wherein the predetermined limit is approximately 1:800.

14. The wound treatment system of claim 11, wherein the wound contact film layer comprises a plurality of fenestrations extending therethrough.

15. The wound treatment system of claim 11, wherein wound contact film layer comprises a plurality of holes extending therethrough.

16. The wound treatment system of claim 11, wherein the wound contact film layer allows air to flow through the wound contact film layer and the pouch allows air to flow through the pouch such that the surgical wound and the absorbent pouch assembly are maintained at substantially the same pressure.

17. A method for manufacturing a dressing deployable to treat a surgical wound using negative pressure therapy without exerting a closure force on the surgical wound, the method comprising:
enclosing an absorbent material in a pouch;
coupling a manifold layer to the pouch, the manifold layer configured to substantially prevent medial collapse of the manifold layer when subjected to a negative pressure;
coupling a drape to the pouch such that the pouch and the absorbent material are between the manifold layer and the drape;
coupling a reduced-pressure interface to the drape, the reduced-pressure interface coupleable to a pump configured to provide the negative pressure at the manifold layer.

18. The method of claim 17, comprising forming the manifold layer with a thickness-to-width-to-length ratio of approximately 1:10:25.

19. The method of claim 17, comprising forming the manifold layer with a thickness-to-area ratio of approximately 1:1600.

20. The method of claim 17, comprising forming the manifold layer with a thickness of approximately 6 millimeters.

21. The method of claim 17, comprising providing the manifold layer with silver ions.

22. The method of claim 17, wherein the absorbent material is a superabsorbent material.
